(19) United States
(12) Patent Application Publication
(10) Pub. No.: US 2006/0029989 A1
(43) Pub. Date: Feb. 9, 2006

(54) Targeting cells having Mad2 mutation for treatment and/or prevention of disease

(75) Inventor: Andrew W. Murray, Brookline, MA (US)

Correspondence Address: MCDERMOTT WILL & EMERY LLP, 600 13TH STREET, N.W., WASHINGTON, DC, 20005-3096, US

(73) Assignee: Regents of the University of California, Oakland, CA (US)

(21) Appl. No.: 11/240,370

(22) Filed: Oct. 3, 2005

Related U.S. Application Data
(63) Continuation of application No. 09/677,592, filed on Oct. 3, 2000.
(60) Provisional application No. 60/156,897, filed on Sep. 30, 1999.

(51) Int. Cl. G01N 33/574; A61K 48/00

(52) U.S. Cl. 435/7.23; 514/44

(57) ABSTRACT

The present invention relates to composition and methods of identifying one or more secondary drug targets and their use in the identification of drugs or drug candidates, particularly for the treatment of cancer or cell replication disorders. The yeast-based synthetic lethal screens are used to functionally identify and validate new gene targets to kill cells with defects in cell cycle pathways. These newly identified gene targets can be used to develop new therapeutics to treat cancer, benign cell growth, and yeast infections.

### 1. FIELD OF THE INVENTION

This invention relates to composition and methods of identifying eukaryote genes, which are important in cell life cycle. More specifically it relates to yeast genes and their mammalian counterparts such as human genes that are essential in cell growth and death. Methods and compositions are provided for identifying and treating cell cycle related gene dysfunctions resulting in diseases and clinical conditions such as malignancy and other diseases associated therewith.

### 2. BACKGROUND OF THE INVENTION

Genetic manipulation of the yeast genome provides a convenient model for identifying essential genes required for eukaryotic cell replication, growth, and death. The complete DNA sequence of the yeast Saccharomyces cerevisiae strain S288C was determined through an international collaboration of more than 100 laboratories on April 1996. General information and databases containing the yeast genome of about 6000 genes are available publicly and can be found for example on public websites such as for example: http://www.ncbi.nlm.nih.gov/Yeast; http://genome-www.stanford.edu/Saccharomyces/. Other databases exist as well and these databases and links therein to other websites are equally suitable for the purposes of this invention. The examples include but are not limited to Yeast GenBank (A collection of all GenBank sequences that are derived from Saccharomyces cerevisiae); Yeast Swiss-Prot (The collection of Swiss-Prot protein sequences that are derived from Saccharomyces cerevisiae); YPD (The Yeast Protein Database maintained by Proteome, Inc.), and periodic updates thereof the content of which is incorporated herein by way of reference. Methods of manipulating yeast are well established and are well known to those skilled in the art and can be found in publicly available web sites by using appropriate keywords, e.g., yeast and protocol, among many others.

While the sequence of the majority of all yeast genes is known and mapped on their respective chromosomes it is still difficult to predict the biological function of many of these genes especially those that have no counterparts in other species or fail to reveal a sufficient sequence homology with known genes. In other words determining the sequence of a gene is easier than ascribing a meaningful function to a gene. The function of only half of the 6,000 genes of the yeast is known. Furthermore, even with genes having known functions, it is not obvious how the function or regulation of such a gene may be modified when combined with another gene's regulatory mechanism or product. Thus, the art is still highly unpredictable when it comes to a problem of identifying a gene's known or unknown function in combination with another gene.

In the past this task was accomplished on a case by case basis, whereby investigators used known gene manipulation techniques and screening methods and applied such methods or techniques to each specific gene of interest or to each particular gene combination. Yeast is often selected as a model due to the ease of manipulation and possibility of screening a large number of candidates in a relatively short period of time. Yeast have the highest rate of recombination and gene conversion among organisms tested, which is several orders of magnitude higher than in mammals. In the post-genomic era, serial gene-knockout programs in yeast (which you can tell by tetrad analysis, e.g., http://bioinformatics.weizmann.ac.il/pub/software/mac/mactetrad69.readme) confirm that about 1 in 6 gene products are essential to the life of that cell (their deletion is lethal) under tested conditions. While a reasonable fraction of tested gene products are enzymes, other genes have either unknown function or their function does not fit into apriori postulation.

Several US patents exist which provide an insight into means of screening and identifying yeast genes and their respective functions. For example, incorporated by reference, U.S. Pat. Nos. 5,916,752 and 5,698,686 disclose telomerase compositions and screening methods; U.S. Pat. Nos. 5,821,076 and 5,756,305 disclose essential survival genes; and U.S. Pat. Nos. 5,585,245 and 5,503,977 deal with ubiquitin split protein sensor. A commonly used approach is screening by gene knockout and complementation. The term “complementation” is used herein as a genetic term intended to mean that the subject genetic element is homologous to a mutant genetic element (homologous by at least 40%) such that when introduced into a cell it rescues the cell from the effects of the mutation (see e.g., U.S. Pat. No. 5,527,896). For example, MEC-1 DNA rescues the mec-1 defect in a mec-1, cdc9-8 cell or RAD9 rescues the rad9 defect in a rad9, cdc9-8 cell through complementation process, and the MEC-1 (or RAD9) DNA so capable is referred to as a “complementing cDNA.” Similarly, human CDC34 is homologous with yeast CDC34 and able to complement the mutation in single (or double mutant) cells, e.g., single mutant cells of yeast strain cdc34. For further details and examples see U.S. Pat. No. 5,866,338 to Hartwell, et al., incorporated herein by way of reference. Means of cross-referencing the yeast and human genes are now achievable and can be for example performed using “XREF2” program as found on http://www.ncbi.nlm.nih.gov/XREFdb/, which is incorporated herein by way of reference. These strategies resulted for example in identification DNA replication accessory factors from the yeast Saccharomyces cerevisiae such as DNA polymerase alpha and the human counterpart gene thereof.

In the setting of anti-cancer drug target identification, the primary defect would be a mutation in a gene conserved from yeast to humans that is frequently deregulated in tumors (e.g., overexpression of cyclin). Gene products with mutations that specifically kill cells with the primary defect would constitute putative “secondary drug targets” (that is, secondary to the primary defect) whose inactivation in tumors may yield great therapeutic advantage. In principle, synthetic lethality can also result when there are two mutations that have an additive negative effect on a single essential biological pathway, or when the mutations inactivate two different but functionally overlapping pathways.

MAD2 is known to interact on a protein-protein level with Bet1p, Cdc20p, and Mad1p. MAD2 exists in two pools in vivo: one in a low-molecular-weight fraction and a second in a larger Mad1p-containing fraction. The association with Mad1p is very tight and independent of cell cycle stages. Chen, R. H., Brady, D. M., Smith, D., Murray, A. W., and Hardwick, K. G. The Spindle Checkpoint of Budding Yeast Depends on a Tight Complex between the Mad1 and Mad2 Proteins. Mol Biol Cell 10, 2607-2618 (1999). In addition, Mad2p and Mad3p remain associated with Cdc20p throughout the cell cycle and binding of Mad2p to Cdc20p requires Mad1p. Hwang, L. H., Lau, L. F., Smith, D. L., Mistrot, C. A., Hardwick, K. G., Hwang, E. S., Amon, A., and Murray, A. W. Budding Yeast Cdc20: A Target of the Spindle Checkpoint Science 279, 1041-4 (1998). However, cdc20 mutants resistant to spindle checkpoint do not form associations with Mad1p, Mad2p and Mad3p, Hwang, L. H., Lau, L. F., Smith, D. L., Mistrot, C. A., Hardwick, K. G., Hwang, E. S., Amon, A., and Murray, A. W. Budding Yeast Cdc20: A Target of the Spindle Checkpoint Science 279, 1041-4 (1998), and bind to different phosphorylated isoforms of Mad1p, Chen, R. H., Brady, D. M., Smith, D., Murray, A. W., and Hardwick, K. G. The Spindle Checkpoint of Budding Yeast Depends on a Tight Complex between the Mad1 and Mad2 Proteins. Mol Biol Cell 10, 2607-2618 (1999). MAD2 is involved in genetic interactions with CIN8, CTF13, and KAR3. While little information is available on MAD2's precise function, this gene is believed to be required for cell cycle delay at mitosis and viability in cells treated with low concentrations of nocodazole or benomyl (Wang, Y., and Burke, D. J.) Checkpoint genes required to delay cell division in response to nocodazole respond to impaired kinetochore function in the yeast Saccharomyces cerevisiae. Mol Cell Biol 15, 6838-44 (1995); Li, R., and Murray, A. W. Feedback control of mitosis in budding yeast. Cell 66, 519-31 (1991). Mad1p, Mad2p, Mad3p and Cdc20p form a complex responsible for the spindle checkpoint required for exit from mitosis (Hwang, L. H., Lau, L. F., Smith, D. L., Mistrot, C. A., Hardwick, K. G., Hwang, E. S., Amon, A., and Murray, A. W. Budding Yeast Cdc20: A Target of the Spindle Checkpoint Science 279, 1041-4 (1998)) and is also required for cell cycle delay in ctfl3 mutants; Wang, Y., and Burke, D. J. Checkpoint genes required to delay cell division in response to nocodazole respond to impaired kinetochore function in the yeast Saccharomyces cerevisiae. Mol Cell Biol 15, 6838-44 (1995). Mad1p-Mad2p complex may be involved in regulation of anaphase promoting complex (APC) and association with Mad1p is required for its spindle checkpoint function and for hyperphosphorylation of Mad1p. Chen, R. H., Brady, D. M., Smith, D., Murray, A. W., and Hardwick, K. G. The Spindle Checkpoint of Budding Yeast Depends on a Tight Complex between the Mad1 and Mad2 Proteins. Mol Biol Cell 10, 2607-2618 (1999). Overproduction of Bub1-5p from a GAL promoter causes a mitosis delay and the phenotype is dependent on other checkpoint genes including BUB2, BUB3, MAD1, MAD2, and MAD3 (Farr, K. A., and Hoyt, M. A. Bub1p kinase activates the Saccharomyces cerevisiae spindle assembly checkpoint. Mol Cell Biol 18, 2738-2747 (1998)) which are essential for preventing degradation of Pds1p and for blocking dissociation of Mcd1p from chromosomes after disruption of mitotic spindles with nocodazole, but not for Clb2p degradation or Clb2p-Cdc28p inactivation; Alexandru, G., Zachariae, W., Schleiffer, A., and Nasmyth, K. Sister chromatid separation and chromosome re-duplication are regulated by different mechanisms in response to spindle damage. Embo Journal 18, 2707-2721 (1999). The response to disruption of mitotic spindle by nocodazole may occur through Mad2p-dependent pathway which blocks degradation of Pds1p and separation of sister chromatids, and through Bub2p-dependent pathway which prevents Clb2p degradation and re-replication of DNA, Alexandru, G., Zachariae, W., Schleiffer, A., and Nasmyth, K. Sister chromatid separation and chromosome re-duplication are regulated by different mechanisms in response to spindle damage. Embo Journal 18, 2707-2721 (1999). Analysis of genetic interactions suggests that Mad1p, Mad2p, Mad3p and Bub1p function in the same regulatory pathway, Alexandru, G., Zachariae, W., Schleiffer, A., and Nasmyth, K. Sister chromatid separation and chromosome re-duplication are regulated by different mechanisms in response to spindle damage. Embo Journal 18, 2707-2721 (1999). MAD2 is also noted as being necessary for sgt1-3 mutant G2 delay at the restrictive temperature Kitagawa, K., Skowyra, D., Elledge, S. J., Harper, J. W., and Hieter, P. SGT1 encodes an essential component of the yeast kinetochore assembly pathway and a novel subunit of the SCF ubiquitin ligase complex. Mol Cell 4, 21-33 (1999).

The Yeast MAD2 gene or product thereof has mammalian homologs and analogs such as human mitotic feedback control protein, T-cell receptor alpha, human plasma membrane calcium pump, rat GTPase Rab8b, human Mad2-like protein, spindle assembly checkpoint component, murine mitotic checkpoint component Mad2, spindle assembly checkpoint protein Mad2p, cell cycle checkpoint protein, DNA gyrase A subunit, SCS2, huntingtin, and RNA polymerase II (DNA directed) 220 kD protein. For example, MAD2 has 41% identity to Xenopus homolog and 40% identity to human homolog hsMAD2. Xenopus and human homologs associate with unattached kinetochores at premetaphase and in noncodazole-treated cells and disappear from kinetochores at metaphase in untreated cells (Chen, R. H., Waters, J. C., Salmon, E. D., and Murray, A. W. Association of spindle assembly checkpoint component XMAD2 with unattached kinetochores). Science 274, 242-6 (1996); Li, Y., and Benezra, R. Identification of a human mitotic checkpoint gene: hsMAD2. Science 274, 246-8 (1996).

Mutated forms are known, e.g., MAD2-1 mutation converts codon for Trp94 to a STOP codon, Zervos Mxi1, a protein that specifically interacts with Max to bind Myc-Max recognition sites. Cell 79, 389 (1994). Null allele has the same phenotype as MAD2-1 and extension of S phase in a MAD2 mutant by treatment with a low concentration of hydroxyurea partially suppresses the toxic effect of benomyl, Li, R., and Murray, A. W. Feedback control of mitosis in budding yeast. Cell 66, 519-31 (1991). Furthermore, disruption of mitotic spindle by nocodazole or benomyl normally causes arrest with high levels of Clb2p and Clb3p and high levels of Cdc28p-Clb kinase activity, but in a MAD2 mutant Clb proteins are not stabilized (Minshull, J., Straight, A., Rudner, A. D., Dernburg, A. F., Belmont, A., and Murray, A. W. Protein phosphatase 2A regulates MPF activity and sister chromatid cohesion in budding yeast. Curr Biol 6, 1609-20 (1996); Li, R., and Murray, A. W. Feedback control of mitosis in budding yeast. Cell 66, 519-31 (1991). It has been observed that the null mutant is unable to prevent sister chromatid separation after disruption of ritotic spindles with nocodazole, but is capable of blocking cytokinesis and DNA re-replication, Alexandru, G., Zachariae, W., Schleiffer, A., and Nasmyth, K. Sister chromatid separation and chromosome re-duplication are regulated by different mechanisms in response to spindle damage. Embo Journal 18, 2707-2721 (1999). When null mutant cells are treated with nocodazole, a mitotic spindle-disrupting drug, they show normal degradation of Pds 1p and normal dissociation of Mcd1p from chromosomes, Alexandru, G., Zachariae, W., Schleiffer, A., and Nasmyth, K. Sister chromatid separation and chromosome re-duplication are regulated by different mechanisms in response to spindle damage. Embo Journal 18, 2707-2721 (1999). Generally, null mutants maintain high level of Clb2p-Cdc28p activity and fail to re-replicate their DNA in the presence of nocodazole, Alexandru, G., Zachariae, W., Schleiffer, A., and Nasmyth, K. Sister chromatid separation and chromosome re-duplication are regulated by different mechanisms in response to spindle damage. Embo Journal 18, 2707-2721 (1999). Mutant cells fail to arrest cell cycle prior to mitosis when spindle formation is incomplete which leads to cell death (Chen, R. H., Waters, J. C., Salmon, E. D., and Murray, A. W. Association of spindle assembly checkpoint component XMAD2 with unattached kinetochores. Science 274, 242-6 (1996); Li, R., and Murray, A. W. Feedback control of mitosis in budding yeast. Cell 66, 519-31 (1991), mutants fail to hyperphosphorylate Madlp in presence of microtubule-disrupting agents Hardwick, K. G., and Murray, A. W. Mad1p, a phosphoprotein component of the spindle assembly checkpoint in budding yeast. J Cell Biol 131, 709-20 (1995). Deletion of MAD2 leads to impaired mitotic checkpoint arrest as induced by nocodazole, Fraschini, R., Formenti, E., Lucchini, G., and Piatti, S. Budding yeast Bub2 is localized at spindle pole bodies and activates the mitotic checkpoint via a different pathway from Mad2. J Cell Biol 145, 979-991 (1999), and in mutants treated with benomyl, lethality ensues upon mitosis Li, R., and Murray, A. W. Feedback control of mitosis in budding yeast. Cell 66, 519-31 (1991). While, overproduction of the human homolog hsMAD2 suppresses the thiabedazole sensitivity of cpf1 null mutants, Li, Y., and Benezra, R. Identification of a human mitotic checkpoint gene: hsMAD2. Science 274, 246-8 (1996), the overproduction of the Xenopus homolog XMad2 cannot suppress the benomyl sensitivity of a MAD2 mutant. (Chen, R. H., Waters, J. C., Salmon, E. D., and Murray, A. W. Association of spindle assembly checkpoint component XMAD2 with unattached kinetochores. Science 274, 242-6 (1996)).

Mutants of MAD2 are involved in genetic interactions with other mutated secondary genes (double mutants). For example double mutants with kar3, cln8 or cbf1 have significantly decreased viability relative to single mutants, Hardwick, K. G., Li, R., Mistrot, C., Chen, R. H., Dann, P., Rudner, A., and Murray, A. W. Lesions in many different spindle components activate the spindle checkpoint in the budding yeast Saccharomyces cerevisiae. Genetics 152, 509-518 (1999). Double mutants with cdc23, cdc15 or cdc20 are not more defective than any single mutants, Hardwick, K. G., Li, R., Mistrot, C., Chen, R. H., Dann, P., Rudner, A., and Murray, A. W. Lesions in many different spindle components activate the spindle checkpoint in the budding yeast Saccharomyces cerevisiae. Genetics 152, 509-518 (1999). However, MAD2 bub2 double mutant has more severe checkpoint defect than either single mutant, Fraschini, R., Formenti, E., Lucchini, G., and Piatti, S. Budding yeast Bub2 is localized at spindle pole bodies and activates the mitotic checkpoint via a different pathway from Mad2. J Cell Biol 145, 979-991 (1999). Other double mutants such as MAD2-1 mutation suppresses mitotic delay of ctf7-203 mutant, Skibbens, R. V., Corson, L. B., Koshland, D., and Hieter, P. Ctf7p is essential for sister chromatid cohesion and links mitotic chromosome structure to the DNA replication machinery. Genes And Development 13, 307-319 (1999). ipl1-321 MAD2 double mutants, like MAD2 single mutants, fail to arrest in the cell cycle because of the absence of the chromosome segregation machinery in both, Biggins, S., Severin, F. F., Bhalla, N., Sassoon, I., Hyman, A. A., and Murray, A. W. The conserved protein kinase Ipl1 regulates microtubule binding to kinetochores in budding yeast. Genes And Development 13, 532-544 (1999). Mad2, Bud2 double null mutant is capable of Clb2p and Clb3p proteolysis, DNA re-replication and cytokinesis after disruption of mitotic spindles with nocodazole, Alexandru, G., Zachariae, W., Schleiffer, A., and Nasmyth, K. Sister chromatid separation and chromosome re-duplication are regulated by different mechanisms in response to spindle damage. Embo Journal 18, 2707-2721 (1999), and it seems that re-replication of DNA in MAD2 bub2 double null mutant treated with nocodazole depends on Cdcl4p, Alexandru, G., Zachariae, W., Schleiffer, A., and Nasmyth, K. Sister chromatid separation and chromosome re-duplication are regulated by different mechanisms in response to spindle damage. Embo Journal 18, 2707-2721 (1999). Some mutants such as glc7-10 MAD2 double mutant phenotypically display wild-type DNA content and wild-type spindle elongation and DNA segregation, Sassoon, I., Severin, F. F., Andrews, P. D., Taba, M. R., Kaplan, K. B., Ashford, A. J., Stark, M. J. R., Sorger, P. K., and Hyman, A. A. Regulation of Saccharomyces cerevisiae kinetochores by the type. 1 phosphatase Glc7p. Genes And Development 13, 545-555 (1999). Others such as MAD2 chl1 double mutant have merely slow growth phenotype, Li, R., and Murray, A. W. Feedback control of mitosis in budding yeast. Cell 66, 519-31 (1991). Bub2p and Mad2p are not in the same epistasis group since Bub2p-dependent block of DNA re-replication in nocodazole-treated MAD2 null mutant cells is bypassed by overproduction of Tem1p.

Finally, there are double mutants which cause cell death, e.g., mutant overproducing Mpslp, tub2-403 MAD2 double mutants that lose viability at 14° C. (indicating that tubulin activates the spindle checkpoint), and mps2-1 MAD2 double mutant which lose viability at 37° C., Hardwick, K. G., Li, R., Mistrot, C., Chen, R. H., Dann, P., Rudner, A., and Murray, A. W. Lesions in many different spindle components activate the spindle checkpoint in the budding yeast Saccharomyces cerevisiae. Genetics 152, 509-518 (1999).

In view of the roles of mad 1 and MAD2 in the cell life cycle, additional information relating to these genes is desirable in order to identify new pathways of cancer prevention and treatment.

### 3. SUMMARY OF THE INVENTION

It is therefore an object of this invention to provide yeast mutants as models of tumor cells or other deregulated cells corresponding to various diseases.

By mass screening and genetic analysis, compounds or drug targets are identified that could specifically kill tumor cells. In this context, examples of compounds that simulate the activity of found lethal mutants are provided. Accordingly, therapeutic agents are contemplated, which are developed from the identification of essential genes of eukaryotic organisms. Such an identified gene (hereinafter “secondary gene”) or product thereof serves as a novel target for therapeutics based on a mechanism which is distinct or similar to the mechanisms of existing drugs. Such a compound inhibits the function of a gene product identified by methods disclosed herein, for example, by producing a phenotype or morphology similar to that found in the original mutant strain.

According to one aspect of the invention, a mutant collection is systematically screened to identify genes and/or gene products, which are targets for drugs. For example, a drug may act as an antagonist by binding reversibly, or preferably irreversibly, to the identified gene or gene product target, and thereby impairing its function. Loss of the function (or the synthesis or the complete processing) of the gene product target will result in inhibition of the tumor cell growth, and preferably will result in death of the tumor cells. This aspect includes a method for identifying anti-cancer agents, including the step of exposing a gene product corresponding to the wildtype sequence of a secondary gene identified by methods disclosed herein to the test agent; and selecting agents which impair (preferably, selectively) the function of the gene product.

In a particular embodiment of the invention, the primary gene defect is preferably one found in or associated with a tumor cell or cell affected with cell cycle deregulation. Alternatively, the primary gene defect in the cell provided by the instant method is analogous or homologous to a defect found in or associated with a mammalian or human tumor cell or chromosomally aberrant cell. By “homologous” is meant a direct relationship among a “family” of genes in which certain sequences or domains are strongly conserved among the members of the family. For instance, MAD2 has 41% identity to Xenopus homolog and 40% identity to human homolog hsMAD2. On the other hand, “analogous” genes may serve similar or “analogous” functions, but they are not directly related (i.e., sequences are not conserved among analogous genes). Within this context, such “analogous” genes are referred to as “functionally homologous” by which is meant that the gene product that has a function in a yeast cell that is analogous to a function in a mammalian cell. For example, Xenopus and human homologs associate with unattached kinetochores at premetaphase and in noncodazole-treated cells and disappear from kinetochores at metaphase in untreated cells (Chen, R. H., Waters, J. C., Salmon, E. D., and Murray, A. W. Association of spindle assembly checkpoint component XMAD2 with unattached kinetochores). Science 274, 242-6 (1996); Li, Y., and Benezra, R. Identification of a human mitotic checkpoint gene: hsMAD2. Science 274, 246-8 (1996). Within certain embodiments of the invention analogs and homologs of MAD2 include but are not limited to human mitotic feedback control protein, T-cell receptor alpha, human plasma membrane calcium pump, rat GTPase Rab8b, human Mad2-like protein, spindle assembly checkpoint component, murine mitotic checkpoint component Mad2, spindle assembly checkpoint protein Mad2p, cell cycle checkpoint protein, DNA gyrase A subunit, SCS2, huntingtin, and RNA polymerase II (DNA directed) 220 kD protein.

In the present method, the primary gene defect may result in the alteration, loss, or inhibition of a function, for example, a cellular function. Thus the present method concerns defects in MAD1 and/or MAD2.

Generally, the functions affected can vary widely. The affected functions may include, but are not limited to, the suppression of tumor growth, DNA damage checkpoint, DNA mismatch repair, nucleotide excision repair, O6-methylguanine reversal, double-strand break repair, DNA helicase function, signaling, cell cycle control, or apoptosis. In a particular embodiment of the invention, the cell life cycle function includes, but is not limited to cells having a defective mitotic checkpoint. Such defects may be effectively modeled by primary gene defects in other organisms such as Drosophila.

By the methods of the present invention, it has been found that certain secondary site mutations can be effected, which may turn out to be lethal to the cell harboring the primary gene defect. Such secondary site mutations may be effected, for example, within a gene selected from the group TUB1, CIN8, STU1, SFI1, TUB2, and MPS2.

Double mutants that involve MAD1 or MAD2 and a secondary gene and/or ORF (open reading frame) are also contemplated within the scope of this invention.

The invention also provides a method of identifying a drug that inhibits the growth or replication of a cell having a mutated MAD1, MAD2 gene or an analog or homolog thereof, by contacting a cell having a mutated MAD1, MAD2 gene or an analog or homolog thereof with the drug; and determining whether the drug modulates the activity of a wildtype secondary gene which is synthetically lethal when it is mutated and is present in combination with mutated MAD1, MAD2 gene or an analog or homolog thereof. Without limiting to above genes other preferred secondary genes are equally suitable comprising at least one secondary gene or product thereof is selected from the group consisting of analogs and homologs of, KAR3, CIN8, KIP1, CIK1, CTF19, CBF1, yeast, human cytoplasmic linker protein, human restin, mouse synaptonemal complex protein, mouse testicular protein, human desmoplakin, mouse Rho-associated, rat SCP1 gene product, human desmoplakin I, rat ROK-alpha, bovine Rho-associated kinase, human AH antigen, human non-muscle myosin heavy chain, human NuMA protein, human SP-H antigen, human KIAA0378, human weak similarity to myosin, human KIAA0336, human similar to mouse CC1, rat cardiac myosin heavy chain 5, human endosomal protein and combinations thereof.

A further object of the invention includes the identification of drug or drug candidates. Hence, after the secondary drug target is elucidated, the secondary drug target can be used to screen for a drug or drug candidate that can potentially interact with the secondary drug target, for example to disable its physiological activity. Accordingly, the present invention may provide a drug or drug candidate that interacts with, binds to, or inhibits a particular gene product. Such gene products may include, but are not limited to examples listed supra. It is desirable that the drug or drug candidate exhibit the capacity to inhibit or arrest the growth of a human tumor or benign neoplastic growth. Preferably this assay consists of a control system in which one observes lethality and test system to which a drug is added and its effect is evaluated based on difference between a control and a test system. If a drug enhances lethality or reverses it, this drug is considered as a candidate drug that may be specific in the biochemical pathway controlled by primary and secondary genes. The specificity is further tested by a number of specificity defining tests, e.g., binding capacity to target genes or products thereof, by introducing double mutants which reverse the originally observed effect, in vitro tests for effect on enzymatic activity of gene products, etc., by the primary and secondary genes.

Thus an object of this invention is to provide means of controlling unwanted proliferation or differentiation of eukaryotic cells. The term “unwanted proliferation” refers to proliferation of cells which is undesired, be it due to transformation of the cells. e.g., neoplastic or hyperplastic, for purposes of wound healing for example keloid, treatment of restenosis, infection by eukaryotic pathogens and other unwanted smooth muscle proliferation, cosmetic applications, retinopathy resulting from diabetes, etc. Likewise, the term “unwanted differentiation” refers to an undesirable change in the differentiation of a cell, such as where a differentiated cell reverts to an earlier state and recovers or retains an ability to proliferate un-naturally or in an “unwanted” manner.

The present invention provides methods of treating malignant conditions, such as prostatic cancer, melanoma, adult and pediatric tumors, e.g., brain tumors of glial origin, astrocytoma, Kaposi's sarcoma, lung adenocarcinoma and leukemias, as well as hyperplastic lesions, e.g., benign hyperplastic prostate and papillomas by administering a therapeutically effective amount of subject drug or a pharmaceutically acceptable derivative thereof.

Most preferably, administration of the drug or drug candidate results in death of the tumor cell, reduction in neoplastic tissue and a therapy for the cancer. Without limiting to cancer, other clinical conditions are anticipated as being treatable by compounds of the present invention. These conditions include, for example, cardiovascular conditions like restenosis, in which smooth muscle cells proliferate excessively in blood vessels after angioplasty, potentially leading to restricted blood flow and death.

Without limiting to aforementioned diseases, this invention also provides a means for treating various mycotic or yeast pathogens selected from a group consisting of Candida albicans, Candida stellatoidea, Candida tropicalis, Candida parapsilosis Candida krusei, Candida pseudotropicalis, Candida quillermondii, Candida rugosa, Aspergillus fumigatus, Aspergillus flavus, Aspergillus niger, Aspergillus nidulans, Aspergillus terreus, Rhizopus arrhizus, Rhizopus oryzae, Absidia corymbifera, Absidia ramosa, and Mucor pusillus or combination thereof.

The present invention also embodies diagnostic and prognostic assays, which assess the phenotype and aggressiveness of a disorder by detecting the expression of a MAD1 or MAD2 protein or expression of regulating genes or gene products thereof.

A further object of this invention includes a pharmaceutical composition comprising a drug, in a pharmaceutically acceptable carrier or diluent, which drug selectively interacts with the production of at least one gene product in a cell population that contains at least one primary gene defect, wherein the exposure of the cell population to the drug arrests cell division selectively in the cell population. Such gene products are encoded or regulated by a human gene analogous or homologous to a yeast gene.

A further object of this invention includes means of gene therapy by gene production in a cell population that contains at least one primary gene defect, wherein the exposure of the cell population to the drug arrests cell division selectively in the cell population. Such gene products are encoded or regulated by a human gene analogous or homologous to a yeast gene.

A synthetic lethal screening method is disclosed based on lethality of yeast mutants having a mutation in a primary gene of interest and secondary target genes, which when present in a mutant form and in combination with the primary gene mutation, determine cell proliferation and death regulation. The subject screening assay uses single and double mutants that are capable of complementing or suppressing the function of the primary gene of interest. Experimental examples enabling this invention are provided which identify novel genes involved in human cell cycle control. These genes include but are not limited to yeast TUB1, CIN8, STU1, SFI1, TUB2, MPS2 and others and also include analogs and homologs of these genes. These genes encode proteins involved in aspects that include but are not limited to aspects of microtubule and spindle formation. The subject screening assay uses single and double mutants that are capable of complementing or suppressing the function of the primary gene of interest (e.g., mad1 or mad2). Experimental examples enabling this invention are provided which identify novel genes involved in cell proliferation.

A further object of this invention includes means of using of gene therapy to induce synthetic lethality in a cell containing at least one defect in a primary gene such as MAD1, MAD2, or a homolog or analog thereof. Within the context of this invention, methods of inducing synthetic lethality include introducing a polynucleotide into a cell containing at least one defect in a primary gene such as MAD1, MAD2, or a homolog or analog thereof wherein the action or expression of the polynucleotide results in the perturbation of a secondary gene that results in synthetic lethality of the cell. Perturbation of the secondary gene includes but is not limited to the upregulation, downregulation, elimination or disruption of the secondary gene. Polynucleotides suitable for use in this invention include, but are not limited to triplex forming oligonucleotides, antisense polynucleotides, RNA-I (for review see Nature 402: 128-129, 1999; which is incorporated herein by reference in its entirety) and ribozymes that specifically target the secondary gene. Secondary genes useful in this regard include those stated previously, TUB1, CIN8, STU1, SFI1, TUB2 and MPS2 and their respective analogs and homologs.

The use of antisense polynucleotides and their applications are described generally in, for example, Mol and Van der Knil, eds., Antisense Nucleic Acids and Proteins Fundamentals and Applications, New York, N.Y., 1992, incorporated by reference herein in its entirety. Suitable antisense oligonucleotides are at least 15 nucleotides in length and up to and including the upstream untranslated and associated coding sequences of the secondary gene of choice. As will be evident to one skilled in the art, the optimal length of antisense oligonucleotides is dependent on the strength of the interaction between the antisense oligonucleotides and their complementary sequence on the mRNA, the temperature and ionic environment in which translation takes place, the base sequence of the antisense oligonucleotide, the presence of secondary and tertiary structure in the mRNA and/or in the antisense oligonucleotide and the preferred delivery mode. For example, soluble antisense oligonucleotides have been used to inhibit transcription/translation of a target gene (Ching et al., Proc. Natl. Acad. Sci. USA 86:10006-10010, 1989; Broder et al., Ann. Int. Med. 113: 604-618 (1990); Loreau et al., FEBS Letters 274:53-56 1990; Holcenberg et al., WO91/11535; WO91/09865; WO91/04753; WO90/13641; and EP 386563, each incorporated herein by reference). Suitable target sequences for antisense polynucleotides include intron-exon junctions (to prevent proper splicing), regions in which DNA/RNA hybrids will prevent transport of mRNA from the nucleus to the cytoplasm, initiation factor binding sites, ribosome binding sites, and sites that interfere with ribosome progression. A particularly preferred target region for antisense polynucleotides is the 5′ untranslated region of the secondary gene of choice.

Antisense polynucleotides targeted to the secondary gene of choice may also be prepared by inserting a DNA molecule containing the target polynucleotide sequence into a suitable expression vector such that the DNA molecule is inserted downstream of a promoter in a reverse orientation as compared to the gene itself. The expression vector may then be transduced, transformed or transfected into a suitable cell resulting in the expression of antisense polynucleotides. Alternatively, antisense oligonucleotides may be synthesized using standard manual or automated synthesis techniques.

Synthesized oligonucleotides may be introduced into suitable cells by a variety of means including electroporation (e.g., as described in Yang et al., Nucl. Acids. Res. 23: 2803-2810, 1995), calcium phosphate precipitation, microinjection, poly-L-ornithine/DMSO (Dong et al., Nucl. Acids. Res. 21: 771-772, 1993). The selection of a suitable antisense oligonucleotide administration method will be evident to one skilled in the art. With respect to synthesized oligonucleotides, the stability of antisense oligonucleotide-mRNA hybrids may be increased by the addition of stabilizing agents to the oligonucleotide. Stabilizing agents include intercalating agents that are covalently attached to either or both ends of the oligonucleotide. Oligonucleotides may be made resistant to nucleases by, for example, modifications to the phosphodiester backbone by the introduction of phosphotriesters, phosphonates, phosphorothioates, phosphoroselenoates, phosphoramidates or phosphorodithioates. Oligonucleotides may also be made nuclease resistant by the synthesis of the oligonucleotides with alpha-anomers of the deoxyribonucleotides, as generally described in Mol and Van der Krul (ibid.).

Within another embodiment, polynucleotide-based inhibitors of the present invention include the triplex forming oligonucleotides, sequence-specific DNA binding drugs that interfere with target gene transcription. Triplex-forming oligonucleotides are generally described in Maher, Bioessays 14: 807-815, 1992; Gee et al., Gene 149: 109-114, 1994; Noonberg et al., Gene 149: 123-126, 1994; Song et al., Ann. NY Acad. Sci. 761: 97-108, 1995; Westin et al., Nuc. Acids. Res. 23: 2184-2191, 1995; and Wand and Glazer, J. Biol. Chem. 207: 22595-22901, 1995, each incorporated herein by reference in its entirety. These oligonucleotides form triple helical complexes under physiological conditions on double-stranded DNA, selectively inhibiting gene transcription by physically blocking RNA polymerase or transcription factor access to the DNA template. See also, e.g., WO 95/25818; WO 95/20404; WO 94/15616; WO 94/04550; and WO 93/09788, each of which is incorporated herein by reference. The triplex forming oligonucleotides may contain either a nucleotide or non-nucleotide tail to enhance the inhibition of transcription factor binding. Within one example, the triplex forming oligonucleotides are targeted to the secondary genes: TUB1, CIN8, STU1, SFI1, TUB2 and MPS2 and their respective analogs and homologs.

For polynucleotide-based inhibitors, the choice of a suitable sequence will be guided by, for example, the type of inhibitor (i.e., triplex forming oligonucleotide or antisense polynucleotide) and the species to be treated. It may be preferable to choose sequences that are conserved between species to permit use in readily available animal models.

The present invention also provides compositions and methods for using ribozyme inhibitors for inhibiting secondary gene expression. The ribozymes can be administered in a variety of ways, including by gene therapy targeted to a desired cell. A ribozyme of the invention is a targets the RNA transcripts of the gene of interest in a sequence-specific manner. For example, ribozymes may be designed to specifically inhibit the transcription of the secondary gene Each ribozyme molecule is designed to contain a catalytically active segment capable of cleaving secondary gene RNA, and further comprises flanking sequences having a nucleotide sequence complementary to portions of the targeted RNA. The flanking sequences serve to anneal the ribozyme to the RNA in a site-specific manner. Absolute complimentary of the flanking sequences to the target sequence is not necessary, however, as only an amount of complimentary sufficient to form a duplex with the target RNA and to allow the catalytically active segment of the ribozyme to cleave at the target sites is necessary. Thus, only sufficient complimentary to permit the ribozyme to be hybridizable with the target RNA under physiological conditions is required. As used herein, the term “ribozyme” means an RNA molecule having an enzymatic activity that is able to cleave or splice other separate RNA molecules in a nucleotide base sequence specific manner. By reference to catalytic or enzymatic RNA molecule is meant an RNA molecule which has complementarity in a substrate binding region to a specific RNA target (e.g. TUB1, CIN8, STU1, SFI1, TUB2 or MPS2 RNA), and also has enzymatic activity that is active to cleave and/or splice RNA in that target, thereby altering the target molecule. In preferred embodiments of the present invention the enzymatic RNA molecule is formed in a hammerhead motif, but the ribozyme may also be formed in the motif of a hairpin, hepatitis delta virus, group I intron or RNAse P RNA (in association with an RNA guide sequence). Examples of hammerhead motifs are described by Rossi et al., AIDS Res. Hum. Retrovir. 8: 183, 1992, hairpin motifs are described by Hampel et al., Biochem. 28:4929, 1989 and Hampel et al., Nucl. Acids Res. 18: 299, 1990, the hepatitis delta virus motif is exemplified in Perrotta and Been, Biochem. 31: 16, 1992, an RNAseP motif is described in Guerrier-Takada et al., Cell 35: 849, 1983, and examples of the group I intron motif are described in Cech et al., U.S. Pat. No. 4,987,071, each of the foregoing disclosures being incorporated herein by reference. These specific motifs are not limiting in the present invention and those of skill in the art will recognize that an enzymatic RNA molecule of the invention has a specific substrate binding site which is complementary to one or more of the target RNA regions and that it has nucleotide sequences within or surrounding that substrate binding site which impart an RNA cleaving activity to the molecule.

The flanking sequences upstream and downstream of the ribozyme catalytic site may comprise segments of any length that effectively imparts the desired degree of targeting specificity for the ribozyme. Preferably a flanking sequence comprises from about 4 to about 24 nucleotides, more preferably from about 6 to about 15 nucleotides, and typically about 9 to 12 nucleotides, and results in base pairing to the substrate sequence immediately upstream and downstream of the RNA sequences which comprise the cleavage site. Polynucleotide inhibitors, e.g., triplex forming oligonucleotides, antisense oligonucleotide, ribozyme, etc., or a combination of such inhibitors targeted to different portions of the target DNA or corresponding RNA can be delivered in a wide variety of ways to targeted cells to facilitate inhibition of the gene of interest. The oligonucleotides can be administered as synthetic oligonucleotides or expressed from an expression vector. The oligonucleotide can be administered ex vivo, i.e., contacted with target cells that have been removed from an individual or other cell source, treated and returned, or the oligonucleotide molecule can be administered in vivo. When administered ex vivo typically the target cells are exposed to mitogens, e.g., serum mitogens (SCF, IL-3, EPO, TPO, etc.) or the like depending on the particular cell population.

These and other objects of the present invention will be evident from the disclosure provided herewith.

### 4. DETAILED DESCRIPTION OF THE INVENTION

This invention provides yeast mutants as models of cancer and other diseases associated with deregulation of cell growth and replication. Yeast is an extremely useful model for studying human diseases. For example, as of Jan. 20, 1996 the following human diseases and clinically important conditions have been identified as having matches between human genes and S. cerevisiae genes/proteins: Hereditary Non-polyposis Colon Cancer; Cystic Fibrosis; Wilson Disease; Glycerol Kinase Deficiency; Adrenoleukodystrophy; Ataxia Telangiectasia; Amyotrophic Lateral Sclerosis; Myotonic Dystrophy; Lowe Syndrome; Neurofibromatosis, Type 1; Choroideremia; Diastrophic Dysplasia; Lissencephaly; Thomsen Disease; Wilms Tumor; Achondroplasia; Menkes Syndrome; Multiple Endocrine Neoplasia 2A; Wiskott-Aldrich Syndrome; Duchenne Muscular Dystrophy; Aniridia; Gonadal Dysgenesis; Breast and Ovarian Cancer, Early Onset; Chronic Granulomatous Disease; Epidermolytic Palmoplantar Keratoderma; Waardenburg Syndrome; Adenomatous Polyposis Coli; Neurofibromatosis, Type 2; Kallmann Syndrome; Tuberous Sclerosis; Polycystic Kidney Disease; Aarskog-Scott Syndrome; Marfan Syndrome; Huntington Disease; Spinocerebellar Ataxia; Long QT Syndrome, Type 1; Fragile X Syndrome; Emery-Dreifuss Muscular Dystrophy; Retinoblastoma; McLeod Syndrome; Norrie Disease; Von Hippel-Lindau Disease; Alzheimer Disease; Hyperekplexia; Agammaglobulinemia, X-linked (for detailed information see http://www.ncbi.nlm.nih.gov/Bassett/Yeast/ and updates thereof as incorporated herein by way of reference). Means of cross-referencing the yeast and human genes are now achievable and can be for example performed using “XREF2” program as found on http://www.ncbi.nlm.nih.zov/XREFdb/, which is incorporated herein by way of reference. Abnormalities in components of the cell cycle surveillance system have been identified in human cancers and other diseases such as listed above. These abnormalities include alterations in cyclin (80-90% of tumors), p53 (50-60% of tumors), and DNA mismatch repair (10-20% of some tumor types such as colon and pancreatic). Often the primary genetic alteration is a loss of function and so a drug discovery program focused on these defects would require restoring the lost function. An alternative approach is to identify which other protein(s) when inhibited selectively kill cells that have the primary defect.

General information and databases containing yeast genome are available publicly and can be found for example on public websites such as for example: http://bioinformatics.weizmann.ac.il; http://ourworld.compuserve.com/homepages/C_Velten/yeast.htm; http://www.ncbi.nlm.nih.gov/Yeast; http://genome-www.stanford.edu/Saccharomyces/; genome-ftp.stanford.edu (directory/yeast/genome_seq); http://vectordb.atcg.com/vectordb/; http://www.mpimg-berlin-dahlem.mpi.de/˜andy/GN/S.cerevisiae/; or http://www.mips. biochem.mpg.de/proj/yeast, the content of which and links therein are incorporated herein by way of reference. Other databases exist as well and these databases and links therein to other websites are equally suitable for the purposes of this invention. The examples include but are not limited to Yeast GenBank (A collection of all GenBank sequences that are derived from Saccharomyces cerevisiae); Yeast Swiss-Prot (The collection of Swiss-Prot protein sequences that are derived from Saccharomyces cerevisiae); YPD (The Yeast Protein Database maintained by Proteome, Inc.), and periodic updates thereof the content of which is incorporated herein by way of reference. Methods of manipulating yeast are well established and are well known to those skilled in the art and can be found in publicly available web sites such as for example www.goshen.edu/bio/veast, www.fhcrc.org/˜gottschling, and www.sacs.ucsf.edu/home/HerskowitzLab, among many others.

The invention features a method of identifying mutant organisms having conditional-sensitive lethal mutations, and subsequently gene products thereof. The disclosed methods are useful for high-throughput screening of genomic or mutant libraries to rapidly identify genes, and corresponding gene products, which are essential for survival. A lethal mutation results in a gene or a protein which is not functional under restrictive conditions (i.e., in a tumor cell). A non-functional gene can have a defect in the promoter resulting in a reduced or abnormal gene expression. A non-functional protein may have a conformational defect causing improper protein folding or abnormal protein degradation. Improper protein folding can result in partial or total failure to fold, to recognize a native substrate, and/or to bind and release the substrate.

The invention pertains to novel compounds that are capable of inhibiting certain genes, such that the inhibited gene is incompatable with a mutated MAD1 or MAD2. Thus, a preferred use for the compounds of the invention is for inhibiting cell proliferation. In particular, the compounds of the invention can be used for treating a subject having an excessive or abnormal cell growth.

There are a wide variety of pathological cell proliferative conditions for which the compounds of the present invention can provide therapeutic benefits, with the general strategy being the inhibition of an anomalous cell proliferation. To illustrate, cell types which exhibit pathological or abnormal growth include various cancers and leukemias, psoriasis, bone diseases, fibroproliferative disorders such as involving connective tissues, atherosclerosis and other smooth muscle proliferative disorders, as well as chronic inflammation.

There are a wide variety of pathological cell proliferative conditions for which the compounds of the present invention can provide therapeutic benefits, with the general strategy being the inhibition of an anomalous cell proliferation. To illustrate, cell types which exhibit pathological or abnormal growth include various cancers and leukemias, psoriasis, bone diseases, fibroproliferative disorders such as involving connective tissues, atherosclerosis and other smooth muscle proliferative disorders, as well as chronic inflammation. Within this context, agents that mimic synthetic lethality found with secondary mutations that are lethal in combination with mutations in MAD2, MAD1, analogs or homologs thereof may be useful in the treatment of human T-cell leukemia (Jin et al. Cell 93: 81-91, 1998).

In addition to proliferative disorders, the treatment of differentiative disorders which result from, for example, de-differentiation of tissue which may (optionally) be accompanied by abortive reentry into mitosis. Such degenerative disorders include chronic neurodegenerative diseases of the nervous system, including Alzheimer's disease, Parkinson's disease, Huntington's chorea, amylotrophic lateral sclerosis and the like, as well as spinocerebellar degenerations. Other differentiative disorders include, for example, disorders associated with connective tissue, such as may occur due to de-differentiation of chondrocytes or osteocytes, as well as vascular disorders which involve de-differentiation of endothelial tissue and smooth muscle cells, gastric ulcers characterized by degenerative changes in glandular cells, and renal conditions marked by failure to differentiate, e.g. Wilm's tumors.

This invention is also directed to the treatment of diseases selected from the group consisting of apudoma, choristoma, branchioma, malignant carcinoid syndrome, carcinoid heart disease, carcinoma (e.g., Walker, basal cell, basosquamous, Brown-Pearce, ductal, Ehrlich tumor, Krebs 2, merkel cell, mucinous, non-small cell lung, oat cell, papillary, scirrhous, bronchiolar, bronchogenic, squamous cell, and transitional cell), histiocytic disorders, leukemia (e.g., B-cell, mixed-cell, null-cell, T-cell, T-cell chronic, HTLV-II-associated, lymphocytic acute, lymphocytic chronic, mastcell, and myeloid), histiocytosis malignant, Hodgkin's disease, immunoproliferative small, non-Hodgkin's lymphoma, plasmacytoma, reticuloendotheliosis, melanoma, chondroblastoma, chondroma, chondrosarcoma, fibroma, fibrosarcoma, giant cell tumors, histiocytoma, lipoma, liposarcoma, mesothelioma, myxoma, myxosarcoma, osteoma, osteosarcoma, Ewing's sarcoma, synovioma, adenofibroma, adenolymphoma, carcinosarcoma, chordoma, craniopharyngioma, dysgerminoma, hamartoma, mesenchymoma, mesonephroma, myosarcoma, ameloblastoma, cementoma, odontoma, teratoma, thymoma, trophoblastic tumor, adenocarcinoma, adenoma, cholangioma, cholesteatoma, cylindroma, cystadenocarcinoma, cystadenoma, granulosa cell tumor, gynandroblastoma, hepatoma, hidradenoma, islet cell tumor, Leydig cell tumor, papilloma, sertoli cell tumor, theca cell tumor, leiomyoma, leiomyosarcoma, myoblastoma, myoma, myosarcoma, rhabdomyoma, rhabdomyosarcoma, ependymoma, ganglioneuroma, glioma, medulloblastoma, meningioma, neurilemmoma, neuroblastoma, neuroepithelioma, neurofibroma, neuroma, paraganglioma, paraganglioma nonchromaffin, angiokeratoma, angiolymphoid hyperplasia with eosinophilia, angioma sclerosing, angiomatosis, glomangioma, hemangioendothelioma, hemangioma, hemangiopericytoma, hemangiosarcoma, lymphangioma, lymphangiomyoma, lymphangiosarcoma, pinealoma, carcinosarcoma, chondrosarcoma, cystosarcoma phyllodes, fibrosarcoma, hemangiosarcoma, leiomyosarcoma, leukosarcoma, liposarcoma, lymphangiosarcoma, myosarcoma, myxosarcoma, ovarian carcinoma, rhabdomyosarcoma, sarcoma, neurofibromatosis, cervical dysplasia, fibrosis, benign prostate hyperplasia, atherosclerosis, restenosis, glomulerosclerosis, cheloid, psoriasis, lentigo, keratosis, achrochordon, molluscum contagiosum, venereal warts, sebaceous hyperplasia, condylomata acuminatum, angioma, venous lakes, chondrodermatitis, granuloma pyogenicum, hidradenitis suppurativa, keloids, keratoacanthoma, leukoplakia, steatocystoma multiplex, trichiasis, superficial epithelial nevus, polyp, junctional nevus, pyogenic granuloma, prurigo nodularis, dermatofibroma, adenoma sebaceum, papilloma, and combinations thereof.

In addition to therapeutic applications (e.g., for both human and veterinary uses) it will be apparent the subject compounds can be used as a cell culture additive for controlling proliferative and/or differentiation states of cells in vitro, for instance, by controlling mitotic checkpoint.

It will also be apparent that differential screening assays can be used to select for those compounds of the present invention with specificity for non-human yeast enzymes. Thus, compounds that act specifically on eukaryotic pathogens; e.g., anti-fungal or anti-parasitic agents, can be selected from the subject inhibitors. To illustrate, inhibitors of the invention can be used in the treatment of candidiasis—an opportunistic infection that commonly occurs in debilitated and immunosuppressed patients. These same inhibitors could be used to treat these infections in patients with leukemias and lymphomas, in people who are receiving immunosuppressive therapy, and in patients with such predisposing factors as diabetes mellitus or AIDS, where fungal infections are a particular problem.

The identification of genes that are synthetic lethal in combination with mutations in the primary gene MAD1, MAD2, or a homolog or analog thereof permit the development of screens for agents that can mimic the secondary mutation. Such agents are useful therapeutics within the context of this invention for the treatment of diseases associated with aberrant proliferation. The secondary genes of the present invention are used, for example, within assays to identify therapeutic agents for the treatment of proliferative diseases such as cancer wherein the disease is characterized by a mutation in the primary gene, such as mutations in MAD1, MAD2 or an analog or homolog thereof. Within these aspects of the invention, screening assays may be carried out to identify agents, including candidate drugs, that modulate the activity of the secondary gene or the secondary gene product or an analog or homolog thereof, wherein such modulation results in the inhibition or reduction in activity of secondary gene product, the up-regulation, down-regulation, elimination or disruption of said secondary gene. Assays in this context may use whole cells expressing the target gene, cell lysates containing the target gene product or may use purified target gene product. Within certain assays of the invention, test agents are incubated with reaction mixtures containing target gene-expressing cells under suitable conditions and for a time sufficient to permit the test agent to modulate the activity of the target gene product and wherein a control sample is incubated under identical conditions in the absence of the test agent. As used herein, modulation of the activity of a target gene product includes, but is not limited to, increasing or decreasing the activity of a target gene product through, for example, direct binding to the gene product and increasing or decreasing the activity of a target gene product by up-regulating or down-regulating transcription or translation of the target gene product. Aspects of the invention include high-throughput screening assays designed to identify modulators of the target gene product.

Within one assay of the invention, candidate drugs are identified by the following method a) exposing a test cell system comprising a cell having a deletion or mutation in a primary gene such as MAD1, MAD2 to a candidate drug; b) comparing the viability of the test cell system with the viability of a control cell system comprising a cell having a deletion or mutation in a primary gene such as MAD1, MAD2 or analogs or homologs thereof and a secondary gene which in its mutant form is synthetically lethal in combination with the primary mutation, such as MAD1, MAD2 or analogs or homologs thereof, wherein a candidate drug that causes the test cell system to be less viable than the control cell system is a therapeutic drug candidate. Within the context of this invention, viability is measured as the ability of a cell to reproduce itself. Non-viable cells are those cells that do not or cannot divide, those that are arrested in any phase of the cell cycle, those that apoptose or proceed along the apoptotic pathway and those that die. Means for determining the presence of viable and non-viable cells will be evident to one skilled in the art and include vital dye staining, measurement of chromosome condensation, and the like.

Within another embodiment is a method of identifying a compound useful in the treatment of proliferative diseases, such as cancer, characterized by the a mutation in the MAD1, MAD2 gene or analog or homolog thereof which comprises the steps of 1) contacting the wild-type gene product of a secondary gene that in its mutant form is synthetically lethal in combination with the mutant MAD1, MAD2 gene or an analog or homolog thereof with a test compound under conditions and for a time sufficient to permit the test compound to effect the gene product; 2) comparing the activity of the gene product in the presence of the test compound with the activity of the gene product in the absence of the test compound. Within this exemplary assay, compounds that can decrease the activity secondary gene product or an analog or homolog thereof are identified as useful for the treatment of cancer or other proliferative diseases characterized by a mutation in the MAD1, MAD2 gene or analog or homolog thereof. Within another embodiment of the invention, whole cell assays are conducted wherein a cell with a wildtype secondary gene or analog or homolog thereof is contacted with a test compound and the expression and/or activity of the tol gene product or analog or homolog thereof is assessed and compared with the expression and/or activity of the wild-type secondary gene product in cells not exposed to the test compound. Within this aspect, those compounds that result in decreased expression or activity of the gene product are useful for the treatment of cancer or other proliferative diseases characterized by a mutation in the MAD1, MAD2 gene or analog or homolog thereof.

For purposes of the assays described herein, assays for modulation of the gene products of the secondary genes are known to those skilled in the art. As would be evident to one skilled in the art, the secondary gene target will determine the appropriate assay for measuring modulation of protein activity as the artisan will be familiar with the assays used to characterize the genes and their respective mutations. For example, the increase or decrease in enzymatic activity of ribonucleotide reductase (the gene products of RNR1 and RNR2) by the direct reduction of ribonucleotides to deoxyribonucleotides using the methods of Thelander et al. (J. Biol. Chem. 255: 7426-7432, 1980) and Engstrom et al., (Biochemistry 18: 2941-2948, 1979), the measurement of thymidilate kinase (the gene product of CDC8) activity can be carried out by measuring the phosphorylation or deoxythumidine monophosphate as described, for example, by Yong and Campbell (J. Biol. Chem. 259: 14394-14398, 1984), and the endo- and 5′ exonuclease activities of the RAD27 protein and the polymerase activities associated with POL1 can be determined using such assays that follow nuclease and polymerase activities. Activity of a protein can also be determined by measuring the increase or decrease in binding of a gene product to its ligand or substrate by, for example, visualization using antibody staining or by immunoprecipitation. While, structural proteins, such as tubulin (the gene products of TUB1 and TUB2) can be measured by measuring the quality and quantity of tubulin made by a cell.

By way of illustration, the assays described in the art can be used to screen for agents which may ultimately be useful for inhibiting at least one fungus implicated in such mycosis as candidiasis, aspergillosis, mucormycosis, blastomycosis, geotrichosis, cryptococcosis, chromoblastomycosis, coccidiodomycosis, conidiosporosis, histoplasmosis, maduromycosis, rhinosporidosis, nocaidiosis, para-actinomycosis, penicilliosis, monoliasis, or sporotrichosis.

Table 1 depicts a synthetic lethal screening strategy for identifying secondary targets. The Table, below, illustrates some of the secondary site mutations found to be lethal in combination with the indicated primary gene defect (the human homologue is also indicated). Once secondary targets have been identified in the model systems, there are several conditions which must be met before it is reasonable to initiate high-throughput screens for inhibitors of the mammalian homologs of these gene products. It is first necessary to validate that the synthetic lethality also occurs in mammalian cells (both matched pair cell lines and tumor cell lines) in which the primary and secondary targets are inactivated. This will require the use of mammalian inducible gene disruption techniques such as ribozymes, antisense molecules, or dominant-negative strategies. The pharmacological feasibility of each putative drug target must be determined simultaneously, since the secondary targets most amenable to the inhibition by small molecules (for example, enzymes with well-defined substrates) will be obvious first choices for further analysis. Only after these tests have been completed can the standard high-throughput screens for inhibitors of these validated mammalian secondary targets be initiated.

TABLE 1
Yeast Strain: MAD2

| | | Found | | | | |
|---|---|---|---|---|---|---|
| | | Using | Yeast | | | Human |
| | | Genetic | Gene | Human | | Gene |
| 1° | 2° | Screen | Essential | Homologs | Function | Essential |
| MAD2 | kar3 | Yes | Yes | Kinesin | -directed microtubule | |
| | | | | family | motor | |
| | cin8 | No | No | Kinesin | microtubule assoc. | No |
| | | | | family | motor | |
| | kip1 | Yes | No | Kinesin | microtubule assoc. | No |
| | | | | family | motor | |
| | cik1 | No | No | | spindle integrity | |
| | ctf19 | Yes | No | H ctf 19 | chromosome segregation | No |
| | cbf1 | Yes | No | USF family | centromere binding/ | No |
| | | | | | transcription factor | |

In addition to such therapeutic uses, anti-fungal agents developed with such differential screening assays can be used, for example, as preservatives in foodstuff, feed supplement for promoting weight gain in livestock, or in disinfectant formulations for treatment of non-living matter, e.g., for decontaminating hospital equipment and rooms.

In similar fashion, side by side comparison of inhibition of a mammalian gene and an insect gene, such as the Drosophilia, will permit selection amongst the subject derivatives of inhibitors which discriminate between the human/mammalian and insect enzymes. Accordingly, the present invention expressly contemplates the use and formulations of the subject in insecticides, such as for use in management of insects like the fruit fly.

In yet another embodiment, certain of the subject inhibitor compounds can be selected on the basis of inhibitory specificity for plant gene relative to the mammalian counterpart. For example, a plant gene can be disposed in a differential screen with one or more of the human enzymes to select those compounds of greatest selectivity for inhibiting the plant enzyme. Thus, the present invention specifically contemplates formulations of the subject inhibitors for agricultural applications, such as in the form of a defoliant or the like.

In another aspect, the present invention provides pharmaceutically acceptable compositions that comprise a therapeutically-effective amount of one or more of the compounds described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intravaginally or intrarectally, for example, as a pessary, cream or foam.

The phrase “therapeutically-effective amount” as used herein means that amount of a compound, material, or composition comprising a compound of the present invention which is effective for producing some desired therapeutic effect by modulating the activity of expression of a target secondary gene in at least a sub-population of cells in an animal containing a mutation in the primary gene (MAD1, MAD2 or an analog or homolog thereof) and thereby blocking the biological consequences of that protein activity in the treated cells, at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As set out above, certain embodiments of the present check point proteins and other proteins mentioned hereinabove and hereinafter may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, eye drops, powders, implants and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The preparations of the present invention may be given orally, parenterally, topically, or rectally. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These peptides and compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compounds employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound that is the lowest dose effective to produce a therapeutic effect. Such an effective dose will generally depend upon the factors described above. Generally, intravenous, intracerebroventricular and subcutaneous doses of the compounds of this invention for a patient, when used for the indicated analgesic effects, will range from about 0.0001 to about 100 mg per kilogram of body weight per day.

If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical formulation (composition).

Defects in the mitotic checkpoint can lead to chromosomal loss, perturbation of the cell cycle and hyperploidy. These defects can play a role in carcinogensis. Mad1 and MAD2 are genes which assist in controlling the mitotic checkpoint. Thus targeting cells with mutations in MAD1 or MAD2 is a viable strategy in cancer prevention.

Genes, mutations in which are incompatible with the mutations in MAD1 and/or MAD2, present potential drug targets for the compounds that can specifically exterminate cells with defective mitotic checkpoint, sparing those cells having functional mitotic checkpoint. Mutations in such genes are synthetically lethal with MAD1 and/or MAD2.

The invention now being generally described, it will be more readily understood by reference to the following examples which are included merely for purposes of illustration of certain aspects and embodiments of the present invention, and are not intended to limit the invention.

### EXAMPLE 1

### Genes which are Synthetically Lethal with MAD1 Mutation

The following strains were tested:
Slm1-1 tub1, MAD1, ade2, ade3, ura3, leu2, trp1, his3
PMAD1, ADE3, URA3
Slm1-162 tub1, MAD1, ade2, ade3, ura3, leu2, trp1, his3
PMAD1, ADE3, URA3
Slm1-289 tub1, MAD1, ade2, ade3, ura3, leu2, trp1, his3
PMAD1, ADE3, URA3
Slm1-303 tub1, MAD1, ade2, ade3, ura3, leu2, trp1, his3
PMAD1, ADE3, URA3
Slm247 cin8, MAD1, ade2, ade3, ura3, leu2, trp1, his3
PMAD1, ADE3, URA3
Slm2-86 cin8, MAD1, ade2, ade3, ura3, leu2, trp1, his3
PMAD1, ADE3, URA3
Slm3-60 sfi1, MAD1, ade2, ade3, ura3, leu2, trp1, his3
PMAD1, ADE3, URA3
Slm3-65 sfi1, MAD1, ade2, ade3, ura3, leu2, trp1, his3
PMAD1, ADE3, URA3
Slm3-120 sfi1, MAD1, ade2, ade3, ura3, leu2, trp1, his3
PMAD1, ADE3, URA3
Slm3-229 sfi1, MAD1, ade2, ade3, ura3, leu2, trp1, his3
PMAD1, ADE3, URA3
Slm3-273 sfi1, MAD1, ade2, ade3, ura3, leu2, trp1, his3
PMAD1, ADE3, URA3
Slm4-145 stu1, MAD1, ade2, ade3, ura3, leu2, trp1, his3
PMAD1, ADE3, URA3
Slm4-176 stu1, MAD1, ade2, ade3, ura3, leu2, trp1, his3
PMAD1, ADE3, URA3

Thus, mutations in the four genes (TUB 1, CIN8, STU1, and SFI1) are synthetically lethal with a MAD1 mutation. That is, these strains are unable to grow without the MAD1 plasmid (pMAD1, ADE3, URA3), unless they are transformed with the TUB1, CIN8, SFI1 or STU1 plasmids, respectfully. TUB1 encodes alpha tubulin. CIN8 encodes a kinesin-related microtubule motor protein. STU1 encodes a microtubule associated protein. SFI1 encodes a novel protein.

### EXAMPLE 2

### Screening for Genes which are Synthetially Lethal with MAD2

A plasmid is introduced in the yeast cells mutant for the MAD2 gene. This plasmid allows the expression and transposition of a marked retrotransposition element called Ty1. After the transposition across the genome, the majority of the cells have one or several copies of the transposon, randomly inserted in the genome. If in a cell a copy of the transposon inserted into a gene, inactivation of which is synthetically lethal with MAD2, such cell will cease division, because both MAD2 and the secondary gene are inactivated in this cell. We find such transpositions by growing out the cells for several generations. The cells that bear such transpositions and do not divide are quickly lost in the population of actively dividing cells. After the growing out selection scheme, we survey the whole genome for the absence of transposition in selected genes. Since transposition is a random process, the absence of such transposition in a certain gene makes this gene a candidate for a synthetic lethal.

This experiment is controlled by a parallel selection in the cells with the functional MAD2. In such cells, transposition should occur in the candidate synthetic lethal (secondary) genes, because the cells with the functional MAD2 will survive the grow out selection.

The novel candidate synthetic lethal genes with MAD2 are then confirmed genetically.

### EXAMPLE 3

### Defects in Microtubule Polymerization, Spindle Pole Bodies, Microtubule Motors, and Kinetochores all Arrest Cells in Mitosis by Activating the Spindle Checkpoint

MATERIALS AND METHODS: For a detailed description of materials and methods see Hardwick, K.G., Li, R., Mistrot, C., Chen, R., Dann, P., Rudner, A., and Murray, A. Lesions in Many Different Spindle Components Activate the Spindle Checkpoint in the Budding Yeast Saccharomyces cerevisiae Genetics 152: 509-518 (1999), which is herein incorporated by reference in its entirety. All strains used are isogenic to W303 (MATa ade2-1 can1-100 ura3-1, leu2-3, 112his3-11, 15 trp1-1, except for the mps2-1 and tub2403 strains and their derivatives, which are isogenic to S288C.

To investigate the types of mitotic lesions that activate the MAD-dependent checkpoint, the interaction of mad-mutations with mutations that cause defects in the execution of particular steps of mitosis are studied. These include mutations that inhibit microtubule depolymerization, prevent spindle pole body duplication, remove centromere-binding proteins, and cdc mutations that arrest cells in mitosis at 370. Mitotic mutations are tested in two classes, conditional lethal mutations and null mutations in nonessential genes. To determine the interaction of mad mutations with cdc mutations that cause arrest in C2 or mitosis, the properties of the mad cdc double mutant are compared with that of the cdc mutant alone. If the cell cycle arrest point of the cdc mutant is independent of the spindle checkpoint, the cdc mad double behaves exactly like the cdc mutant alone: the cell cycle arrests at the nonpermissive temperature, and the single and double mutants lose viability at the same rate when incubated at the permissive temperature. In contrast, if the arrest of the cdc mutant depends on the spindle checkpoint, inactivation of the checkpoint will allow the mad cdc double mutants to pass through mitosis at the restrictive temperature even though the spindle is defective. Thus, combining such cdc mutations with mad mutations will produce double mutants that fail to arrest in mitosis and lose viability faster than the cdc mutant alone when the strains are incubated at the nonpermissive temperature.

### EXAMPLE 4

### Synthetic Lethal Screening based on Inducible Expression of the Analyzed Gene

Experiments are performed by crossing a haploid lacking a microtubule motor to a mad strain. In a preferred embodiment, experiments are performed by using gene disruption to create heterozygosity for loss of a microtubule motor gene in a strain already heterozygous for a mad mutation. This latter method eliminates the possibility that slowly growing strains such as kar3 will have acquired suppressor mutations, it is however the more cumbersome method.

The hybrids that contain recessive synthetic lethal mutations are crossed with the wild type strain, sporulated and dissected, and the spore tetrads are checked for the appropriate single mutation pattern of segregation of the synthetic lethal phenotype. The mutants that show such pattern of segregation are cloned by complementation. Variations of this and other screening yeast methods are known (see e.g., U.S. Pat. Nos. 5,912,154; 5,908,752; 5,876,951; 5,869,287; 5,866,338; 5,789,184; 5,674,996; 5,578,477; 5,527,896; 5,352,581; 5,175,091; and 5,139,936) and one skilled in the art readily knows which one select for a specific purpose.

### EXAMPLE 5

### Complementation of a Yeast Strain with a Human cDNA Library

In this example the principle is provided whereby human cDNAs are identified, which encode proteins that substitute above described synthetic lethality in yeast with aberrant MAD1 or MAD2. A yeast strain is constructed which contains the aberrant MAD1 or MAD2. A cDNA library using mRNA prepared from the human fetal fibroblasts is constructed in a S. cerevisae vector containing the constitutive yeast promoter such as ADH for expression of the human cDNAs (see for details e.g., U.S. Pat. Nos. 5,783,661; 5,952,195). The library is transfected into host MAD1 or MAD2 yeast strain and 105 independent transformants are screened, by replica plating, for their ability to grow on glucose or galactose. Several transformants are isolated as a result whose growth patterns are rescued by expression of a human cDNA. Some of them are already known genes, e.g., human homolog of CDCl₄, ABL1 a human homolog of Abelson murine leukemia viral oncogene, fibroblast growth factor receptor bfr-2, but some are less known and appears to be related or “homologous” to genes such as an epithelial cell receptor in the eph/elk family of protein kinases, calcium channel protein, or human Wee1 kinase.

### EXAMPLE 6

### Screening for Pharmaceutical Antagonists and Agonists

Drugs useful in treating cell cycle or DNA replication disorders identified in the present invention can be now screened using established yeast models as described hereinabove. Several classes of drugs can be screened such as chemical, organic and inorganic compounds, peptides or peptide mimetics, antisense molecules, antibodies, etc. Methods of generating and performing high-throughput screening of chemical libraries are well established and are well known to those skilled in the art. Using the insights gained in the cell cycle regulation a drug discovery program is established to screen selected candidates in yeast-based assays and evaluate their therapeutic potential by further testing mammalian cells or cell lines. In essence assay consists of control system in which one observes lethality and test system to which a drug is added and its effect is evaluated based on difference between control and test system. If drug enhances lethality or reverses it this drug is considered as a candidate drug that may be specific in the biochemical pathway controlled by primary and secondary genes. The specificity is further tested by a number of specificity defining tests, e.g., binding capacity to target genes or products thereof, by introducing double mutants which reverse originally observed effect, in vitro tests for effect on enzymatic activity of gene products, etc. These pharmaceutical agents would ideally inhibit aspects of the cell cycle in a predictable fashion, thereby enabling the determination of their future promise in cancer or other cell cycle related clinical conditions. Among tested compounds several are found as being active at pharmaceutically acceptable concentrations. These include but are not limited to: triButyrate (4-phenylbutyrtic acid sodium salt or sodium phenylbutyrate), various benzopyran drivatives as prepared by known in the art means. See, for example, U.S. Pat. Nos. 5,359,115; 5,362,899; 5,288,514; 5,733,920 or PCT publications WO 94/08051; WO92/10092; WO93/09668; WO91/07087; WO93/20242. Accordingly, a variety of libraries on the order of 1000 to 100,000 or more diversomers of the subject compounds can be synthesized, and, by use of a high throughput assay for detecting inhibitors, such as described in PCT publication WO 94/09135, rapidly screened for biological activity. For a review of methods of combinatorial synthesis, and methods of library screening and deconvolution, see, e.g., E. M. Gordon et al. (1994) J. Med. Chem. 37:1385-1401, and references cited therein.

### EXAMPLE 7

### Treatment of Clinical Conditions Due to Benign or Malignant Cell Proliferation

Drugs that are screened out as positive in yeast based assays are then tested for treating diseases caused by excessive cell growth. Most of such diseases are malignant diseases, i.e., cancers of any of a wide variety of types, including without limitation, solid tumors and leukemias such as apudoma, choristoma, branchioma, malignant carcinoid syndrome, carcinoid heart disease, carcinoma (e.g., Walker, basal cell, basosquamous, Brown-Pearce, ductal, Ehrlich tumor, Krebs 2, merkel cell, mucinous, non-small cell lung, oat cell, papillary, scirrhous, bronchiolar, bronchogenic, squamous cell, and transitional cell), histiocytic disorders, leukemia (e.g., B-cell, mixed-cell, null-cell, T-cell, T-cell chronic, HTLV-II-associated, lymphocytic acute, lymphocytic chronic, mast-cell, and myeloid), histiocytosis malignant, Hodgkin's disease, immunoproliferative small, non-Hodgkin's lymphoma, plasmacytoma, reticuloendotheliosis, melanoma, chondroblastoma, chondroma, chondrosarcoma, fibroma, fibrosarcoma, giant cell tumors, histiocytoma, lipoma, liposarcoma, mesothelioma, myxoma, myxosarcoma, osteoma, osteosarcoma, Ewing's sarcoma, synovioma, adenofibroma, adenolymphoma, carcinosarcoma, chordoma, craniopharyngioma, dysgerminoma, hamartoma, mesenchymoma, mesonephroma, myosarcoma, ameloblastoma, cementoma, odontoma, teratoma, thymoma, trophoblastic tumor, adenocarcinoma, adenoma, cholangioma, cholesteatoma, cylindroma, cystadenocarcinoma, cystadenoma, granulosa cell tumor, gynandroblastoma, hepatoma, hidradenoma, islet cell tumor, Leydig cell tumor, papilloma, sertoli cell tumor, theca cell tumor, leiomyoma, leiomyosarcoma, myoblastoma, myoma, myosarcoma, rhabdomyoma, rhabdomyosarcoma, ependymoma, ganglioneuroma, glioma, medulloblastoma, meningioma, neurilemmoma, neuroblastoma, neuroepithelioma, neurofibroma, neuroma, paraganglioma, paraganglioma nonchromaffin, angiokeratoma, angiolymphoid hyperplasia with eosinophilia, angioma sclerosing, angiomatosis, glomangioma, hemangioendothelioma, hemangioma, hemangiopericytoma, hemangiosarcoma, lymphangioma, lymphangiomyoma, lymphangiosarcoma, pinealoma, carcinosarcoma, chondrosarcoma, cystosarcoma phyllodes, fibrosarcoma, hemangiosarcoma, leiomyosarcoma, leukosarcoma, liposarcoma, lymphangiosarcoma, myosarcoma, myxosarcoma, ovarian carcinoma, rhabdomyosarcoma, sarcomas, neoplasms (e.g., bone, breast, digestive system, colorectal, liver, pancreatic, pituitary, testicular, orbital, head and neck, central nervous system, acoustic, pelvic, respiratory tract, and urogenital), neurofibromatosis, and cervical dysplasia.

Some diseases occur due to excessive but benign cell proliferation (i.e. non-malignant). Examples of such diseases are fibrosis, benign prostate hyperplasia, atherosclerosis, restenosis, glomulerosclerosis, cheloid, psoriasis, lentigo, keratosis, achrochordon, molluscum contagiosum, venereal warts, sebaceous hyperplasia, condylomata acuminatum, angioma, venous lakes, chondrodermatitis, granuloma pyogenicum, hidradenitis suppurativa, keloids, keratoacanthoma, leukoplakia, steatocystoma multiplex, trichiasis, superficial epithelial nevus, polyp, junctional nevus, pyogenic granuloma, prurigo nodularis, dermatofibroma, adenoma sebaceum, and other diseases of the skin and non-malignant neoplastic diseases such as for example Kaposi's sarcoma, papilloma.

The effect of sodium phenylbutyrate or benzopyran derivatives is then determined on the tumorigenic phenotype of human glioblastoma cells in vivo. Fisher 344 rats received an inoculation of syngeneic 9 L gliosarcoma cells (4×10⁴) into the deep white matter of the right cerebral hemisphere. The animals are then subjected to two weeks of continuous treatment with sodium phenylbutyrate (550 mg/kg/day, s.c.), using osmotic minipumps transplanted subcutaneously. In control rats the minipumps are filled with saline. Statistical analysis of data employed the Fisher's Exact Test. Treatment of glioblastoma cells with the drug results in time-and dose-dependent growth arrest accompanied by similarly diminished DNA synthesis. After 4-6 days of continuous treatment with 4 mM phenylbutyrate, there is approximately 50% inhibition of growth. Phenylbutyrate, which is an intermediate metabolite of phenylacetate inhibited tumor cell replication, while the end metabolite, phenylacetylglutamine, is inactive. In addition to inducing selective tumor cytostasis, both phenylacetate and phenylbutyrate promoted cell maturation and reversion to a nonmalignant phenotype, manifested by an altered pattern of cytoskeletal intermediate filaments, loss of anchorage-independence, and reduced tumorigenicity in athymic mice. These profound changes in tumor behavior are accompanied by alterations in the expression of cyclin genes implicated in growth control, angiogenesis, and immunosuppression.

All of the above-cited internet sources, patents, publications, and references within are hereby expressly incorporated by way of reference in their respective entireties.

The invention being thus described, it will be obvious that the same may be varied in many ways. Such variations are not to be regarded as a departure from the spirit and scope of the invention, and all such modifications are intended to be included within the scope of the following claims.

What is claimed is:

1. A method of identifying a drug that inhibits the growth or replication of a cell having a mutated MAD2 gene or an analog or homolog thereof, said method comprising the steps of:
(a) contacting a cell having a mutated MAD2 gene or an analog or homolog thereof with the drug; and
(b) determining whether the drug modulates the activity of a wildtype secondary gene which is synthetically lethal when it is mutated and is present in combination with mutated MAD2 gene or an analog or homolog thereof.

2. The method of claim 1 in which the cell is a tumor cell.

3. The method of claim 1, comprising the further step of comparing the results of step (b) with a control cell grown without the drug.

4. The method of claim 3 in which the control cell is a normal cell.

5. The method of claim 3 in which the control cell is a tumor cell.

6. The method of claim 3 in which the control cell has a mutated MAD2 gene or an analog or homolog thereof.

7. The method of claim 1 in which the secondary gene is selected from the group consisting of analogs and homologs TUB1, CIN8, SFI1, STU1 and combinations thereof.

8. A method of identifying a compound useful in the treatment of tumor cells having a mutated MAD2 gene or homolog or analog thereof which comprises the steps of:
(a) contacting a secondary gene product, which in its mutant form is synthetically lethal in combination with a mutated MAD2 gene or homolog or analog thereof, with a test compound; and
(b) determining the activity of the secondary gene product.

9. The method of claim 8, comprising the further step of comparing the activity of the secondary gene product in the presence of the test compound with the activity of the gene product in the absence of the secondary gene product.

10. The method of claim 8 in which the secondary gene is selected from the group consisting of analogs and homologs TUB1, CIN8, SFI1, STU1 and combinations thereof.

11. A screening assay system for identifying a drug, comprising:
(a) the control yeast cell having a deletion in MAD2 gene, analog or homolog thereof and a wildtype secondary gene in which its mutant form is synthetically lethal in combination with a deletion of MAD2;
(b) a test yeast cell system comprising the drug and the yeast with the MAD2 deletion; and
(c) a detector for comparing the viability of MAD2 in the test system compared to the control system where a decrease in viability of the test cell system identifies positive drug candidates.

12. A method of screening for the presence of benign or malignant cell growth in a tissue sample comprising the steps of:
a) providing a tissue sample from an individual suspected of having cancer;
b) assessing the expression of MAD2 in the cells of the tissue sample; and
c) comparing the MAD2 expression of the tissue sample with the MAD2 expression of a control sample, wherein the presence of aberrant expression of MAD2 in the test sample as compared with expression in the control sample is indicative of the presence of carcinoma.

13. A pharmaceutical composition comprising an effective amount of a drug and a pharmaceutically acceptable carrier or diluent, said drug capable of selectively interacting with at least one secondary gene or gene product in a target cell which comprises a mutated or deleted primary MAD2 gene or gene product, whereby the exposure of the target cell to the drug arrests cell division or selectively kills cells.

14. The pharmaceutical composition of claim 13 wherein said drug comprises oligonucleotide, gene product, homologs or analogs of oligonucleotide or gene product, a small molecule, or a peptide mimetic.

15. A method for treating a human or animal hosting or susceptible of hosting a disease associated with MAD2 mutation which comprises providing the pharmaceutical composition of claim 13 and treating said human or animal with an effective amount of the pharmaceutical composition.

16. The method of claim 13 in which the disease is selected from the group consisting of apudoma, choristoma, branchioma, malignant carcinoid syndrome, carcinoid heart disease, carcinoma (e.g., Walker, basal cell, basosquamous, Brown-Pearce, ductal, Ehrlich tumor, Krebs 2, merkel cell, mucinous, non-small cell lung, oat cell, papillary, scirrhous, bronchiolar, bronchogenic, squamous cell, and transitional cell), histiocytic disorders, leukemia (e.g., B-cell, mixed-cell, null-cell, T-cell, T-cell chronic, HTLV-II-associated, lymphocytic acute, lymphocytic chronic, mast-cell, and myeloid), histiocytosis malignant, Hodgkin's disease, immunoproliferative small, non-Hodgkin's lymphoma, plasmacytoma, reticuloendotheliosis, melanoma, chondroblastoma, chondroma, chondrosarcoma, fibroma, fibrosarcoma, giant cell tumors, histiocytoma, lipoma, liposarcoma, mesothelioma, myxoma, myxosarcoma, osteoma, osteosarcoma, Ewing's sarcoma, synovioma, adenofibroma, adenolymphoma, carcinosarcoma, chordoma, craniopharyngioma, dysgerminoma, hamartoma, mesenchymoma, mesonephroma, myosarcoma, ameloblastoma, cementoma, odontoma, teratoma, thymoma, trophoblastic tumor, adenocarcinoma, adenoma, cholangioma, cholesteatoma, cylindroma, cystadenocarcinoma, cystadenoma, granulosa cell tumor, gynandroblastoma, hepatoma, hidradenoma, islet cell tumor, Leydig cell tumor, papilloma, sertoli cell tumor, theca cell tumor, leiomyoma, leiomyosarcoma, myoblastoma, myoma, myosarcoma, rhabdomyoma, rhabdomyosarcoma, ependymoma, ganglioneuroma, glioma, medulloblastoma, meningioma, neurilemmoma, neuroblastoma, neuroepithelioma, neurofibroma, neuroma, paraganglioma, paraganglioma nonchromaffin, angiokeratoma, angiolymphoid hyperplasia with eosinophilia, angioma sclerosing, angiomatosis, glomangioma, hemangioendothelioma, hemangioma, hemangiopericytoma, hemangiosarcoma, lymphangioma, lymphangiomyoma, lymphangiosarcoma, pinealoma, carcinosarcoma, chondrosarcoma, cystosarcoma phyllodes, fibrosarcoma, hemangiosarcoma, leiomyosarcoma, leukosarcoma, liposarcoma, lymphangiosarcoma, myosarcoma, myxosarcoma, ovarian carcinoma, rhabdomyosarcoma, sarcoma, neurofibromatosis, cervical dysplasia, fibrosis, benign prostate hyperplasia, atherosclerosis, restenosis, glomulerosclerosis, cheloid, psoriasis, lentigo, keratosis, achrochordon, molluscum contagiosum, venereal warts, sebaceous hyperplasia, condylomata acuminatum, angioma, venous lakes, chondrodermatitis, granuloma pyogenicum, hidradenitis suppurativa, keloids, keratoacanthoma, leukoplakia, steatocystoma multiplex, trichiasis, superficial epithelial nevus, polyp, junctional nevus, pyogenic granuloma, prurigo nodularis, dermatofibroma, adenoma sebaceum, papilloma, and combinations thereof.

17. The method, according to claim 15, wherein the disease comprises yeast infection.

18. The method of claim 15, wherein the disease comprises breast cancer.

19. A method of treating cancer cells having abnormal accumulation of MAD2 gene or an anaolog or homolog thereof which comprises administering a pharmaceutical composition comprising an effective amount of an agent and a pharmaceutically acceptable carrier or diluent, said agent capable of selectively interacting with at least one secondary gene or gene product that is found in a cell having at least one primary gene defect, wherein said gene product is selected from (or wherein said lethal gene codes for) a human isozyme TUB1, CIN8, SFI1, STU1 and combinations thereof.

20. A recombinant eukaryotic cell comprising at least one secondary gene and at least one primary gene MAD2, or analog or homolog thereof, wherein said primary gene MAD2 is mutated such that the up-regulation, down-regulation, elimination, or disruption of said secondary gene results in synthetic lethality.

21. The recombinant eukaryotic cell of claim 20 in which at least one secondary gene or product thereof is selected from the group consisting of analogs and homologs TUB1, CIN8, SFI1, STU1 and combinations thereof.

22. The recombinant eukaryotic cell of claim 20 in which the primary gene is eliminated or disrupted.
